(19) United States
(12) Patent Application Publication
(10) Pub. No.: US 2022/0047846 A1
(43) Pub. Date: Feb. 17, 2022

(54) ROLLING SLEEVE FOR AN ENDOLUMINAL SHAFT

(71) Applicant: Covidien LP, Mansfield, MA (US)

(72) Inventors: Scott J. Prior, Branford, CT (US); Anthony B. Ross, Boulder, CO (US)

(21) Appl. No.: 17/395,328

(22) Filed: Aug. 5, 2021

Related U.S. Application Data
(60) Provisional application No. 63/125,258, filed on Dec. 14, 2020.
(60) Provisional application No. 63/064,938, filed on Aug. 13, 2020.

(51) Int. Cl. A61M 25/01; A61B 1/00; A61B 34/30; A61M 25/10

(52) U.S. Cl. CPC A61M 25/0119; A61B 1/00151; A61B 2034/303; A61B 1/00082; A61M 25/1011; A61B 34/30

(57) ABSTRACT

The present disclosure describes a rolling sleeve having a malleable body. The rolling sleeve is configured to protect an endoluminal shaft from surrounding tissue during insertion, navigation, and/or treatment within a body lumen.

### CROSS-REFERENCE TO RELATED APPLICATIONS

This application claims benefit of and priority to U.S. Provisional Patent Application Nos. 63/125,258 filed Dec. 14, 2020, and 63/064,938 filed Aug. 13, 2020, the disclosure of each of the above-identified applications is hereby incorporated by reference in its entirety.

### BACKGROUND

### Technical Field

The present disclosure is generally related to a sleeve configured to be combined with an endoluminal shaft, and more particularly, a rolling sleeve configured to be combined with an endoluminal shaft, such as an endoscope or catheter.

### Description of Related Art

A wide variety of endoluminal shafts, including endoscopes and catheters, as well as sleeves designed to be used with such devices, have been developed. Of these known devices, each has certain advantages and disadvantages. However, there is an ongoing need to provide alternative endoluminal shafts, endoscopes, catheters, and/or sleeves. For example, some known endoluminal shafts alone or in combination with sleeves affixed thereto may have difficulty maintaining proper alignment through a central portion of a body lumen when inserted, advanced, or navigated therethrough. More particularly, some known endoluminal shafts having sleeves affixed thereto may be unable to maintain proper alignment in a central portion of a body lumen because the sleeve begins to bunch up or fold onto itself as the sleeve is advanced with the shaft. The effect of which may be that the shaft may come into contact with the tissue prior to when needed too. This premature contact often may cause damage to the surrounding tissue, such as may be commonly found from a scrape or puncture wound. In addition, failure to maintain proper central alignment within the body lumen may ultimately place the endoluminal shaft improperly positioned prior to treatment or testing. Thus, there exists a need to provide a sleeve which can be easily combined with an endoluminal shaft to more efficiently align the shaft within the body lumen and is not affixed to the shaft.

### SUMMARY

The present disclosure describes a sleeve configured to be used with any endoluminal shaft. The sleeve is configured to continuously roll over itself. The sleeve is also designed to aid in properly aligning and maintaining the endoluminal shaft in a generally central portion of a body lumen thereby preventing premature contact between the shaft and the tissue defining the body lumen.

In some embodiments, a sleeve, and particularly a rolling sleeve, is described which is configured for positioning on an endoluminal shaft. The rolling sleeve includes a malleable generally tubular body having an exterior portion and an interior portion defining a sealed compartment. The exterior and interior portions are configured to repeatedly invert or switch places as the malleable generally tubular body is moved longitudinally and/or the sleeve is rolled over itself. The tubular body defines a channel therethrough along the interior portion. The channel is configured to receive at least a distal end portion of an endoluminal shaft therein and/or therethrough. The sealed compartment stores a biocompatible fluid maintained therein. The fluid may be any biocompatible liquid or gel.

In some embodiments, the tubular body further includes one or more exterior pleats extending along at least one of the interior or exterior portions of the body. The one or more exterior pleats configured to form one or more external air gaps between the sleeve and the body lumen.

In some embodiments, the tubular body further includes one or more interior pleats extending along at least a portion of the channel, if not all of the channel. The one or more interior pleats configured to form one or more internal air gaps between the sleeve channel and an endoluminal shaft positioned therein.

In some embodiments, the sleeve defines a circular transverse cross-section. In some embodiments, the sleeve defines a non-circular transverse cross-section.

In some embodiments, the sleeves described herein may be configured to be used with a robotic endoluminal shaft.

In some embodiments, the sleeves described herein may be configured to be used with a bronchoscope.

Methods and kits including the rolling sleeves described herein are also provided.

### BRIEF DESCRIPTION OF THE DRAWINGS

Various aspects and features of the present disclosure are described herein below with reference to the drawings, wherein:

FIG. 1A is a perspective view a sleeve as described in at least one embodiment herein;

FIGS. 1B and 1D are side or longitudinal cross-sectional views of the sleeve of FIG. 1A as described in at least one embodiment herein;

FIG. 1C is an end or transverse cross-sectional view of the sleeve of FIG. 1A as described in at least one embodiment herein;

FIGS. 2A, 3A, 4A, and 5A are end or transverse cross-sectional views of various sleeves described in at least one embodiment herein;

FIGS. 2B, 3B, 4B, and 5B are end or transverse cross-sectional views of the various sleeves of FIGS. 2A, 3A, 4A, and 5A, respectively, within a body lumen as described in at least one embodiment herein;

FIGS. 6A and 6B are side or longitudinal cross-sectional views of a sleeve and an endoluminal shaft as described in at least one embodiment herein;

FIGS. 7A and 7B are side or longitudinal cross-sectional views of a sleeve and an endoluminal shaft as described in at least one embodiment herein;

FIGS. 8A-8C are perspective views of a sleeve, an anchor member, and an endoluminal shaft within a body lumen;

FIG. 9A is a perspective view of an Endoflip™ Impedance Planimetry System as described herein;

FIGS. 9B is a schematic view of an endoluminal shaft associated with the Endoflip™ Impedance Planimetry System as described herein;

FIG. 9C is a perspective view of a sleeve in combination with the endoluminal shaft of FIG. 9B as described in at least one embodiment herein;

FIGS. 9D-9F are perspective views of a sleeve and an endoluminal shaft as described in at least one embodiment herein;

FIG. 10 is a top view of kit as described in at least one embodiment herein; and

FIG. 11 is a schematic design of one or more methods of using a sleeve and an endoluminal shaft as described in at least one embodiment herein.

### DETAILED DESCRIPTION

The present disclosure describes a rolling sleeve configured to be used with any endoluminal shaft. The rolling sleeve is designed to prevent premature contact between the shaft and the tissue of a body lumen in which the shaft is placed, i.e., inserted, advanced, navigated, etc. By preventing premature contact between the shaft and the tissue, the rolling sleeve further prevents the shaft, and particularly any equipment associated therewith such as a visualization device or lens, from becoming smudged or dirtied during insertion, advancement, navigation, etc. within the body lumen, which should improve body lumen visualization. The endoluminal shaft may be any endoscopic device suitable for insertion into a natural or artificial lumen defined within a patient. The endoluminal shaft may be configured to be used by a person or by a robotic surgical apparatus.

In some embodiments, the endoluminal shaft is an endoscope, including but not limited to, a sigmoidoscope, colonoscope, anoscope, laparoscope, or bronchoscope. In some embodiments, the endoluminal shaft is a catheter or extended working channel. In some embodiments, the endoluminal shaft is a surgical instrument, such as a locating guide, an imaging device, a guidewire, a surgical balloon, a biopsy tool, a cytology brush, an aspirating needle, or an ablation device.

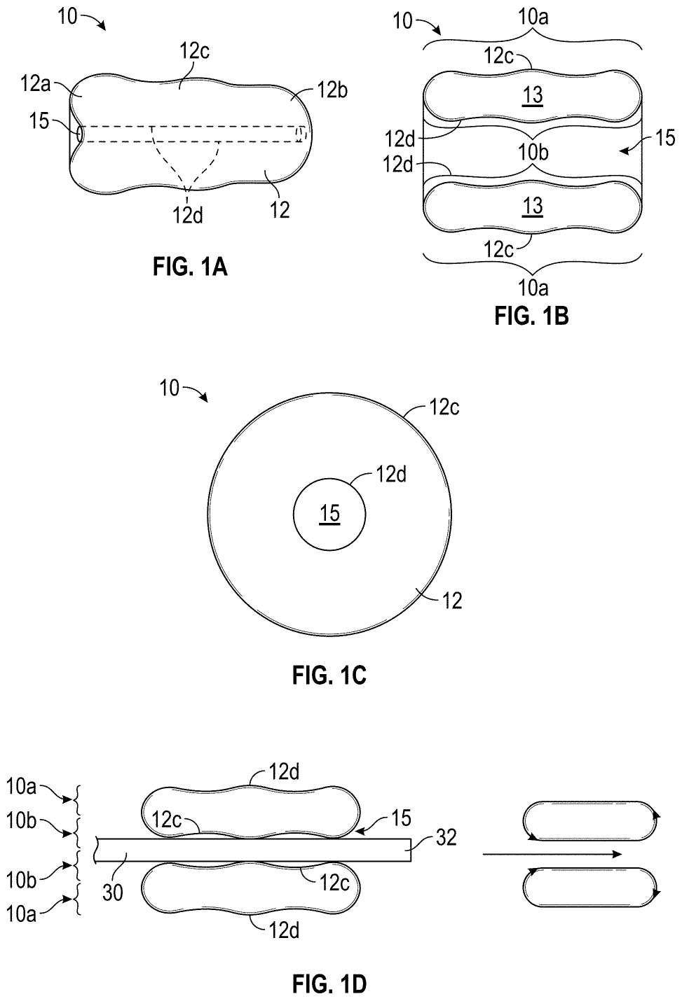

FIGS. 1A-1D depict a rolling sleeve 10 configured for use with an endoluminal shaft 30 as described herein. The rolling sleeve 10 includes a generally tubular body 12 extending between a proximal end portion 12a and a distal end portion 12b. Both the proximal end portion 12a and the distal end portion 12b of the rolling sleeve 10 are configured to be positioned on and/or around a portion of an endoluminal shaft 30. The tubular body 12 also includes a channel 15 extending therethrough configured to receive an endoluminal shaft 30 therein. Both of the body 12 and the channel 15 are malleable.

The generally tubular body 12 includes an exterior portion 12c and an interior portion 12d defining a sealed compartment 13 configured to receive and maintain a biocompatible fluid therein. Initially, prior to rolling, as shown in FIGS. 1A-1C, the exterior portion 12c of the body 12 defines an exterior part 10a of the rolling sleeve 10 and the interior portion 12d of the body 12 defines an interior part 10b of the rolling sleeve 10. The channel 15 extending through the sleeve 10 along the interior part 10b of the sleeve 10, and particularly in some embodiments along the interior portion 12d of the body 12. As shown in FIG. 1D, however, when the sleeve 10 is moved axially along the channel 15, and particularly onto an endoluminal shaft 30, the sleeve 10 rolls over itself (as indicated by the circular arrows) causing the exterior and interior portions 12c, 12d of the tubular body 12 to invert. For example, when advanced axially and/or rolled, the exterior portion 12c of the body 12 transitions from the exterior part 10a to the interior part 10b of the sleeve 10 and the interior portion 12d of the body 12 transitions from the interior part 10a to the exterior part 10b of the sleeve 10. In such embodiments, the interior part 10b of the sleeve 10 continues to define the channel 15 while the interior and exterior portions 12c, 12d of the tubular body 12 define the channel 15 in an alternating or rolling fashion. The sleeve 10 may be continuously rolled over itself and/or the exterior and interior portions 12c, 12d inverted any number of times sufficient to properly position the sleeve 10 onto the endoluminal shaft 30 and/or to properly position the endoluminal shaft 30 in a given body lumen.

The interior part 10b of the sleeve 10 defines an outer perimeter of the channel 15 and is configured to butt up against and/or engage the exterior of the endoluminal shaft 30. The exterior part 10a of the sleeve 10 defines an outer perimeter of the sleeve 10 and is configured to butt up against and/or engage the interior portion of a body lumen. In this configuration, any portion of the endoluminal shaft 30 positioned and/or maintained within the channel 15 of the sleeve 10 is prevented from coming into direct contact with the body lumen.

The sleeve 10 and/or the body 12 is malleable. By malleable, the sleeve 10 and/or body 12 defines a nonspecific generally tubular shape which is pliable and/or may be easily influenced to adapt to forces applied thereto from outside the sleeve 10 and/or body 12.

The sleeve 10 and/or the body 12 may be made of any suitable elastic biocompatible material. The elastic material configured to allow the outer perimeter of the sleeve (and/or tube) and the outer perimeter of the channel to stretch or shrink, as needed to ensure the sleeve rolls upon itself. Some examples include, but are not limited to, biocompatible elastomers such as polyurethane elastomers, polyamide elastomers, polyether amide elastomers, polysiloxane modified styrene-ethylene/butyl block copolymer, polycarbonate-urethane, polycarbonate-urethane cross-linked polyol, silicone elastomer, rubber, silicone rubber, polyether urethane, polyester urethane, polyether polyester copolymer, polypropylene oxide, and combinations thereof.

The sleeve 10 and/or tubular body 12 includes a sealed compartment 13 configured to receive and maintain a biocompatible fluid. The biocompatible fluid may be a liquid or a gel. The fluid may not be a gas. Some non-limiting examples of a biocompatible fluid includes water, saline, dextrose, lactated ringers, hydrogels, ultrasound gel, and combinations thereof.

As shown in FIG. 1C, in some embodiments, the transverse cross-section of the sleeve 10 and/or body 12 may be generally circular. As further shown in FIGS. 1A-1C, in some embodiments, the transverse cross-section of the sleeve channel 15 may be generally circular.

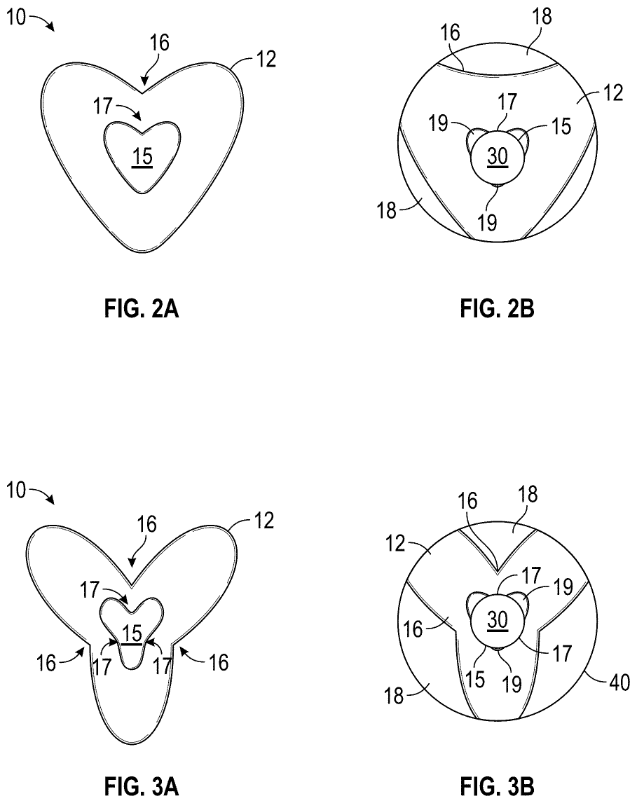

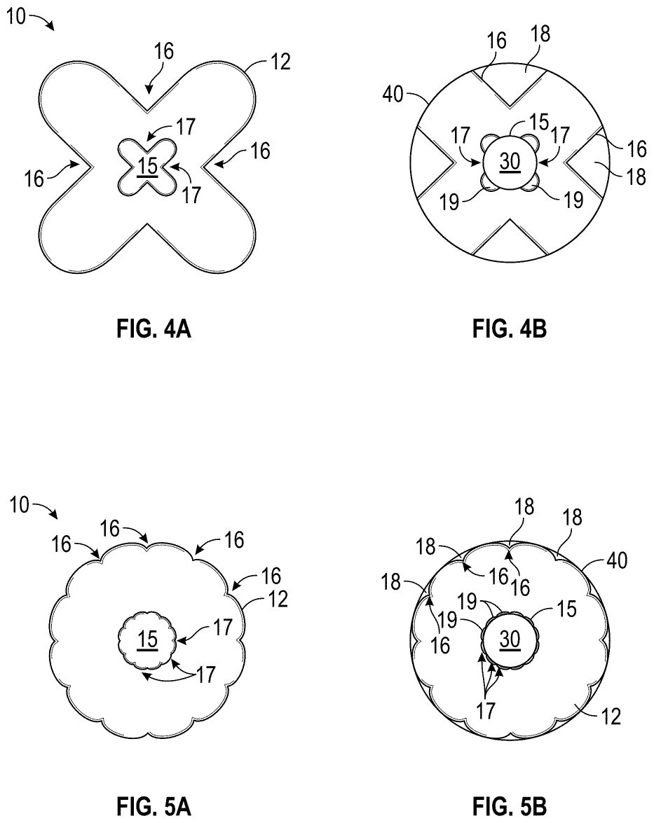

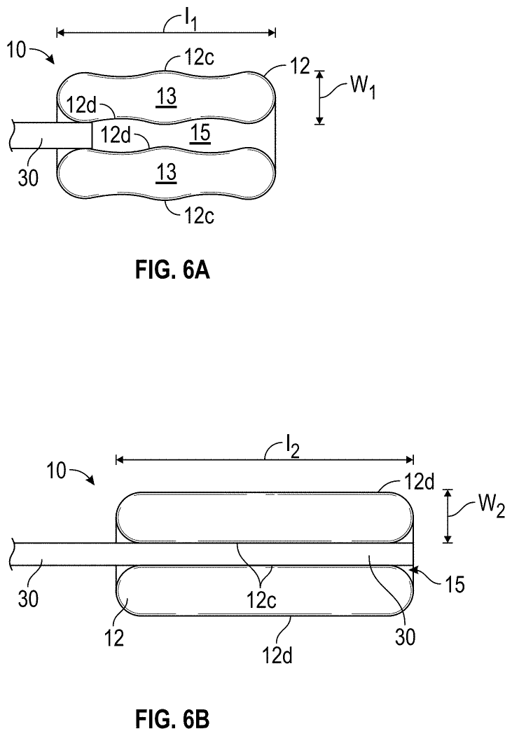

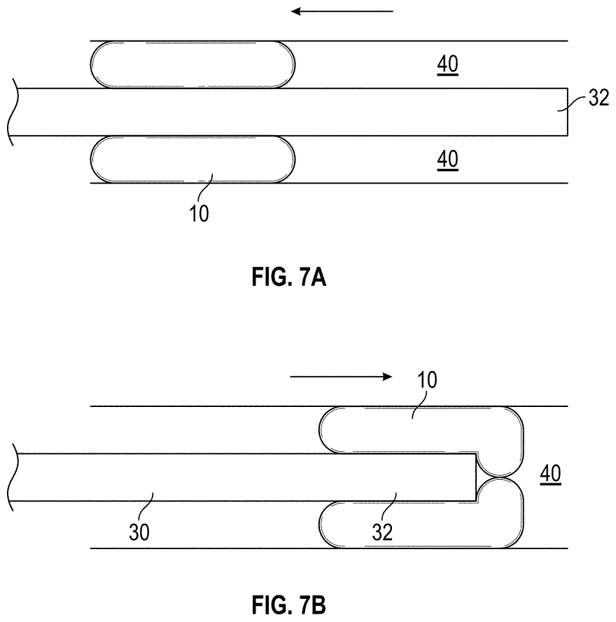

In some embodiments, as shown in FIGS. 2A-5B, the sleeve 10 and/or the tubular body 12 may further include: one or more exterior pleats 16 rendering the transverse cross-section of the exterior of the sleeve 10 and/or tubular body 12 to be generally non-circular; and/or one or more interior pleats 17 rendering the transverse cross-section of the channel 15 to be generally non-circular. For example, in some embodiments, as shown in FIGS. 2A, 3A, 4A, and 5A, the sleeve 10 and/or tubular body 12 may include a one or more exterior pleats 16 extending longitudinally along thereof to form: a generally heart-shaped exterior transverse cross-section (FIG. 2A); a generally Y-shaped exterior transverse cross-section (FIG. 3A); a generally X-shaped exterior transverse cross-section (FIG. 4A); or a generally flower petal-shaped exterior transverse cross-section (FIG. 5A). Additional examples include, in some embodiments, as shown in FIGS. 2A, 3A, 4A, and 5A, the sleeve 10 and/or tubular body 12 may include a one or more interior pleats 17 extending longitudinally along the channel 15 thereof to form: a generally heart-shaped transverse cross-section of the channel 15 (FIG. 2A); a generally Y-shaped transverse cross-section of the channel 15 (FIG. 3A); a generally X-shaped transverse cross-section of the channel 15 (FIG. 4A); or a generally flower petal-shaped transverse cross-section of the channel 15 (FIG. 5A).

In some embodiments, the transverse cross-section of the exterior of the shaft 10 and/or body 12 may define a generally non-circular shape and the transverse cross-section of the channel may define a generally circular shape. In some embodiments, the transverse cross-section of the exterior of the shaft 10 and/or body 12 may define a generally non-circular shape and the transverse cross-section of the channel may define a generally non-circular shape. In some embodiments, the transverse cross-section of the exterior of the shaft 10 and/or body 12 may define a generally circular shape and the transverse cross-section of the channel may define a generally non-circular shape.

As depicted in FIGS. 2B, 3B, 4B, and 5B, when the sleeve 10 and/or tubular body 12 of FIGS. 2A, 3A, 4A, and 5A, respectively, is positioned on an endoluminal shaft 30 and placed into a body lumen 40, the one or more exterior pleats 16 may create one or more external air gaps 18 between an exterior portion of the sleeve 10 (and/or tubular body 12) and the body lumen 40. The one or more external gaps 18 may be spaced intermittently around the exterior of the sleeve 10 and/or body 12. The one or more external air gaps may be beneficial in preventing pressure from building between the sleeve (and/or body 12) with the body lumen and/or pressure building on one side of the body lumen while the sleeve (and/or body 12) is advanced axially, forward or backward, along the endoluminal shaft. In addition, in some embodiments wherein the endoluminal shaft 30, such as a bronchoscope or catheter, is configured to be used in an airway body lumen, the external air gaps 18 may be beneficial to maintain a flow of allow air to continuously pass through and/or around the protective sleeve 10 (and/or body 12) to maintain the flow of oxygen for breathing purposes.

As further depicted in FIGS. 2B, 3B, 4B, and 5B, when the sleeve 10 and/or tubular body 12 of FIGS. 2A, 3A, 4A, and 5A, respectively, is positioned on an endoluminal shaft 30 and placed into a body lumen 40, the one or more interior pleats 17 may form internal air gaps 19 between a portion of the channel 15 and an exterior surface of the endoluminal shaft. The one or more internal gaps 19 may be spaced intermittently around the exterior of the channel 15. The one or more internal air gaps 19 may be beneficial in preventing pressure from building between the interior part of sleeve (and/or body 12) with the endoluminal shaft and/or pressure building on one side of the channel or shaft while the sleeve (and/or body 12) is advanced axially, forward or backward, inside the body lumen. In addition, in some embodiments wherein the endoluminal shaft 30, such as a bronchoscope or catheter, is configured to be used in an airway body lumen, the internal air gaps 19 may be beneficial to maintain a flow of allow air to continuously pass through and/or around the protective sleeve 10 (and/or body 12) to maintain the flow of oxygen for breathing purposes.

As further depicted in FIGS. 2B, 3B, 4B, and 5B, in some embodiments, at least one of the exterior pleats 16 or interior pleats 17, when positioned between the endoluminal shaft 30 and the body tissue surrounding the body lumen 40, may define different sized and/or shaped external or internal air gaps 18, 19. In addition, some of the external and/or internal air gaps 18, 19 may change in size and/or shape, or even open or close, as the sleeve 10 is rolled within the body lumen 40 on the shaft 30.

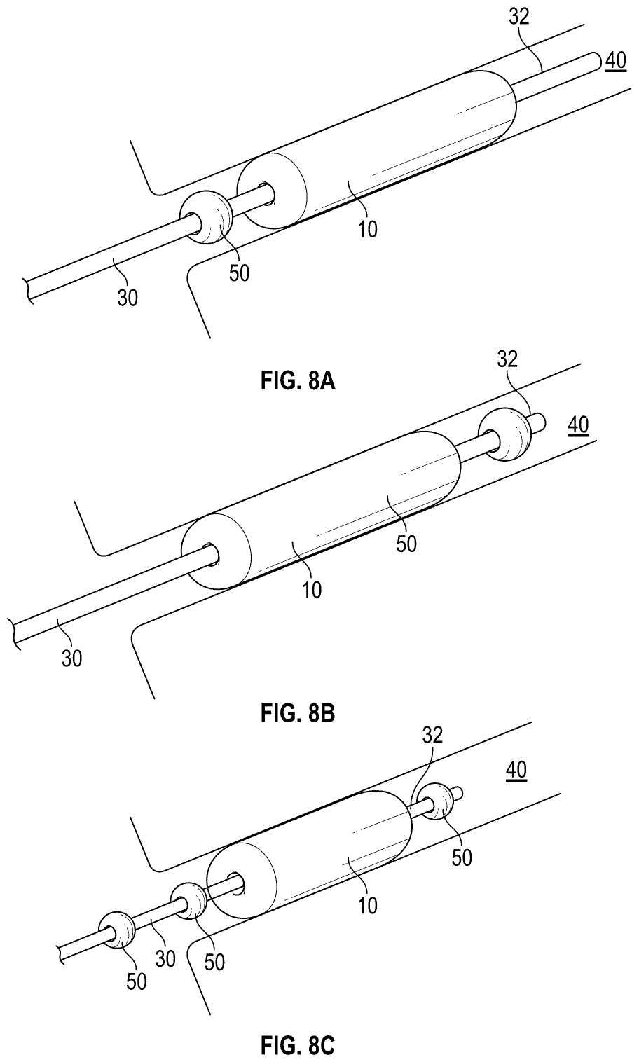

As shown in FIG. 6A-6B, the rolling sleeves 10 described herein are configured to receive an exterior of any endoluminal shaft 30 within a channel 15 defined within the sleeve 10. In FIG. 6A, a distal end portion 32 of an endoluminal shaft 30 is shown being positioned into a proximal end portion 12a of a generally tubular body 12 of rolling sleeve 10, and particularly a channel 15 defined within the generally tubular body 12 of rolling sleeve 10. The generally tubular body 12 includes an exterior portion 12c and an interior portion 12d defining a sealed compartment 13 configured to maintain a biocompatible fluid therein. The exterior and interior portions 12c, 12d are configured to repeatedly invert as the malleable generally tubular body 12 is moved longitudinally and/or axially.

FIG. 6B, the distal end portion 32 of the endoluminal shaft 30 is shown advancing through the channel 15 of the sleeve 10. As the endoluminal shaft 30 advances axially and/or longitudinally through the channel 15, a variety of things may occur including: the channel 15 may expand radially to accommodate the shaft 30 therein due to any radial force applied thereto by the shaft 30; the sleeve 10 (and/or the tubular body 12 and/or the sealed compartment 13) may increase in length, i.e., l₁≤l₂, and decrease in width, i.e., w₁>w₂; and/or the sleeve 10 rolls on itself, i.e., the exterior and interior portions 12c, 12d of the body 12 invert due to rolling of the body 12. In some embodiments, each of these may occur.

In some embodiments, the linear motion (e.g., axially or longitudinally) of the endoluminal shaft 30 may occur at a 1:1 ratio with the advancement of the sleeve 10. In such embodiments, the position of the sleeve 10 on the endoluminal shaft 30 is generally maintained as both the endoluminal shaft 30 and the sleeve 10 are advanced through a body lumen 40 at the same pace while the sleeve 10 rolls over itself.

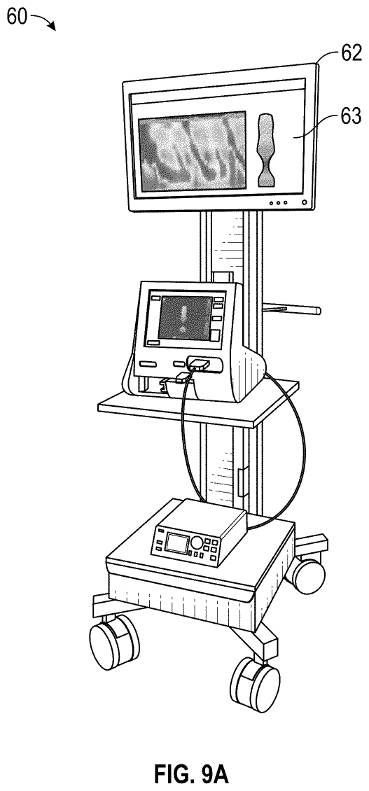

In some embodiments, the linear motion of the endoluminal shaft 30 may not occur at a 1:1 ratio with the advancement of the sleeve 10. In such embodiments, the position of the sleeve 10 on the endoluminal shaft 30 may change as both the sleeve 10 and the endoluminal shaft 30 are advanced through a body lumen 40 at a different pace while the sleeve 10 rolls over itself. For example, in some embodiments, the sleeve 10 may advance at a slower pace than the endoluminal shaft 30 thereby falling behind the distal end portion 32 of the endoluminal shaft 30 as the shaft 30 is advanced in the lumen 40. By falling behind, the sleeve 10 shifts proximally (as indicated by the arrow in FIG. 7A) along the endoluminal shaft 30 farther away from the distal end portion 32 thereof. As illustrated in FIG. 7A, a sleeve 10 which advances slower in the lumen 40 than the shaft 30, potentially exposes a longer portion of the distal end portion 32 of the shaft 30 to the body lumen 40.

In another example, in some embodiments, the sleeve 10 may advance at a faster pace than the endoluminal shaft 30 thereby getting ahead of the distal end portion of the endoluminal shaft 30 as the shaft 30 is advanced in the lumen 40. As shown in FIG. 7B, a sleeve 10 which advances faster in the lumen 40 than the shaft 30 potentially places the sleeve 10 ahead of distal end portion 32 of the shaft 30 within the lumen 40. In such an instance, the malleable sleeve 10 may interfere with the insertion of the shaft 30, as well as potentially block any visual sensors and/or exit ports positioned on the distal end portion 32 of the shaft 30.

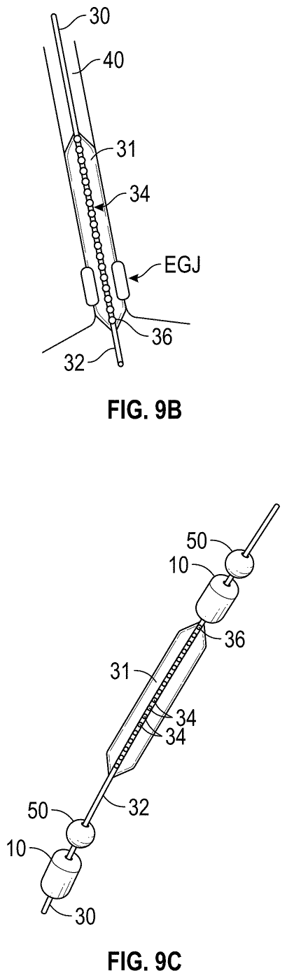

In order to aide in preventing the sleeve 10 from moving proximally or distally out of a predetermined position along the shaft 30, in some embodiments, one or more anchor members 50 may be affixed to the shaft 30. For example, as shown in FIGS. 8A-8C, in some embodiments, one or more anchor members 50 may be: positioned proximal to the sleeve 10 on the shaft 30 to prevent the sleeve 10 from moving out of position proximally while inside the body lumen 40 (FIG. 8A); positioned distal to the sleeve 10 on the shaft 30 to prevent the sleeve 10 from moving out of position distally while inside the body lumen 40 (FIG. 8B); or both (FIG. 8C).

The one or more anchor members 50 are configured to block or stop the sleeve 10 from sliding beyond the anchor members 50. In some embodiments, the one or more anchors 50 may be integrated with the endoluminal shaft 30 as inflatable balloons spaced intermittently across the shaft 30. The anchor members 50 may be made of an elastic material to inflate/deflate. In such embodiments, the anchor members 50 may be individually inflatable and/or deflatable as needed and may be controlled either wirelessly or through the shaft 30. In addition, not only does the anchor member 50 in the inflated configuration prevent the sleeve 10 from passing therethrough, but inflation of the anchor members 50 may also cause the sleeve 10 to advance along the shaft 30 when the inflating anchor member 50 is in direct contact with the sleeve 10.

In some embodiments, an inflatable anchor member may be positioned on both sides of a sleeve 10, as shown in FIG. 8C, wherein during insertion of the shaft 30 into the lumen 40, the first anchor member 50 proximal to the sleeve 10 is inflated and the second anchor member 50 distal the sleeve 10 is deflated, and during withdrawal or retraction of the shaft 30 from the body lumen 40, the first anchor member 50 is deflated and the second anchor member 50 distal the sleeve 10 is inflated to block the sleeve 10 from falling off the end of the shaft 30 as withdrawn.

In some embodiments, the one or more anchor members 50 may be a separate device added to the endoluminal shaft 30 as needed. In such embodiments, the one or more anchors members 50 may be formed of a rigid or semi-rigid biocompatible material, such as a hardened plastic material. The separate anchor members 50 may be configured to either snap on to the shaft 30 or configured to be affixed to the endoluminal shaft 30 by any locking mechanism or other fastening means including, but not limited to, screws, bolts, pins, adhesives, and the like.

Although depicted as generally circular, the one or more anchor members 50 may define any suitable shape including but not limited to elliptical-shaped or polygonal-shaped.

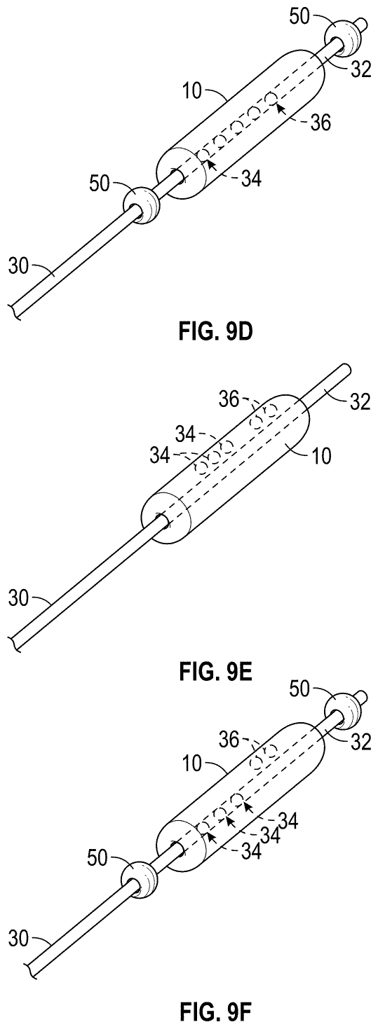

In some embodiments, as shown in FIGS. 9A-9F, the rolling sleeves 10 described herein may be configured to work with Medtronic's Endoflip™ Impedance Planimetry System 60 (FIG. 9A) and the endoluminal shafts 30 associated therewith, i.e., the Endoflip™ measurement catheter and/or the Esoflip™ dilation catheter. The Endoflip™ Impedance Planimetry System is an advanced imaging technology that utilizes at least one of the Endoflip™/Esoflip™ catheters to provide real-time measurements of pressure and dimensions of the gastroesophageal junction during an endoscopic procedure. The system 60 converts the real-time measurements into real-time imaging 63 of the junction which can be displayed on a monitor 62 for review by medical personnel.

As schematically depicted in FIG. 9B, the endoluminal shaft 30 (e.g., Endoflip™ or Esoflip™) associated with the Endoflip™ Impedance Planimetry System 60 may include a dilation balloon 31 that can be inflated/deflated by the infusion of a conductive solution therein, and a plurality of electrodes 34 that measure voltage and/or a plurality of pressure sensors 36 within the balloon 31. It is believed that as the balloon 31 inflates under user control, the Endoflip System uses the voltages measured from the electrodes 34 and the pressure measurements from the pressure sensors 36 to estimate and visually display the diameter of the junction along the measurement area generally defined by the length of the balloon 31.

In some embodiments, the one or more of the rolling sleeves 10 and/or the anchor members 50 described herein may be combined with the endoluminal shafts 30 (e.g., Endoflip™ or Esoflip™) associated with the Endoflip™ Impedance Planimetry System 60. As shown in FIG. 9C, in some embodiments, the rolling sleeve 10 may be positioned on the shaft or catheter 30 on one or both sides of the balloon 31 to avoid with interfering with the inflation and/or deflation of the balloon 31 with the conductive solution. In such embodiments, the one or more rolling sleeves 10 not only prevent premature contact of the shaft 30 with the tissue of the body lumen 40, but also increases the likelihood that the shaft 30 is maintained in the general center of the lumen 40 prior, during, and after the balloon 31 is inflated/deflated. In some embodiments, the one or more anchor members 50 may be positioned at least between the balloon 31 and the sleeve 10.

In some embodiments, the rolling sleeve 10 may be configured to replace the dilation balloon of the endoluminal shafts 30 (e.g., Endoflip™ or Esoflip™) associated with the Endoflip™ Impedance Planimetry System 60. More specifically, as shown in FIGS. 9D-9F, in some embodiments, the shaft 30 may be free of a dilation balloon but include one or more rolling sleeves 10 and/or anchor members 50 described herein. Unlike the dilation balloon 31 of FIGS. 9B-9C, the one or more rolling sleeves 10 does not need to be inflated and/or deflated to insert the catheter or shaft 30 and/or to take measurements because the sleeve 10 is a separate device from the catheter or shaft 30 and is configured to continuously roll over itself. As such, the plurality of electrodes 34 that measure voltage and/or a plurality of pressure sensors 36 described above may be maintained either on the shaft 30, in the rolling sleeve 10, or both. More particularly, the plurality of electrodes 34 that measure voltage and/or a plurality of pressure sensors 36 may: both be maintained with the catheter or shaft 30 and not the sleeve 10, the sleeve 10 configured to be maintained on the catheter or shaft 30 in a position directly over the electrodes and/or sensor (FIG. 9D); both be maintained with the sleeve 10 and not the catheter or shaft 30, the electrodes and/or sensors being wireless (FIG. 9E); or, each of the catheter or shaft 30 and the rolling sleeve 10 include one of the electrodes 34 or sensors 36 (FIG. 9F).

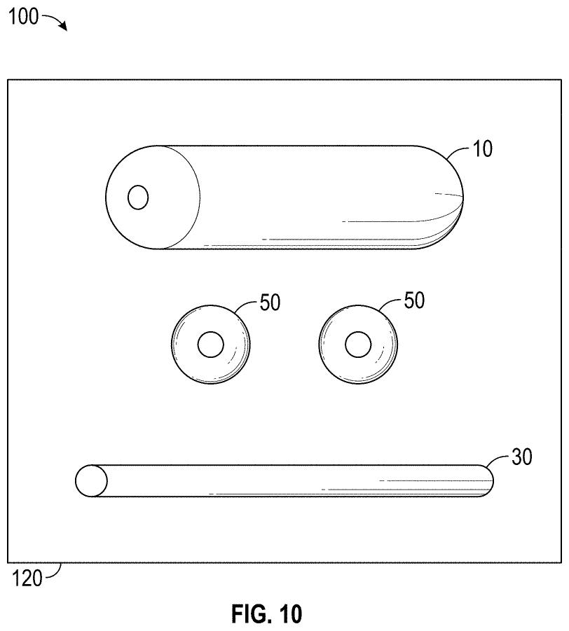

Turning to FIG. 10, one or more of the sleeves 10 described herein, alone or in any combination with the one or more anchor members 50 or endoluminal shafts 30 described herein may be combined to form a kit 100 and/or packaged in a sterile sealable package 120.

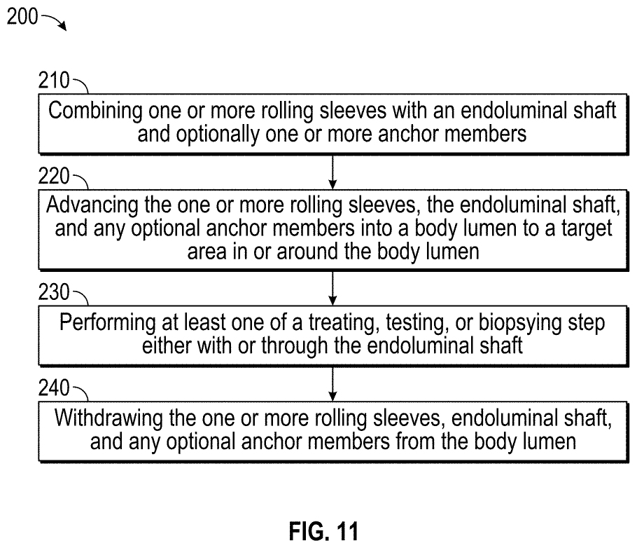

Turning to FIG. 11, one or more of the sleeves described herein, alone or in any combination with the one or more anchor members or endoluminal shafts described herein may also be combined with to perform certain methods. In some embodiments, the methods may be directed to inserting one or more rolling sleeves and an endoluminal shaft into a body lumen 200.

As shown in FIG. 11, initially the methods 200 include combining one or more rolling sleeves with an endoluminal shaft and optionally one or more anchor members 210. The sleeve and the shaft may be combined in any variety of ways. For example, in some embodiments, the sleeve and the shaft may be combined by positioning a distal end portion of an endoluminal shaft into a channel defined within a rolling sleeve. In another example, the channel of the rolling sleeve may be positioned on a distal end portion of an endoluminal shaft and the sleeve rolled over the distal end portion of the shaft.

In some embodiments, the methods may further include combining one or more anchor members with the shaft. As described herein, the anchor members and the shaft may be combined in a variety of ways. For example, in some embodiments, the anchors and the shaft may be combined by positioning one or more anchor members on the distal end portion of the shaft before, after, or both, the sleeve is positioned on the shaft. In another example, the anchor members may be integral the endoluminal shaft prior to positioning of the sleeve.

Once properly combined, the method proceeds to include advancing of the one or more rolling sleeves, the endoluminal shafts, and any optional anchor members into a body lumen to a target are in or around the body lumen. By advancing, the shaft is moved axially in a distal direction thereby advancing the distal end portion of the endoluminal shaft and the one or more protective rolling sheets longitudinally into a body lumen, with or without the one or more anchor members. Such a method causes the sleeve to roll over itself, and particularly the tubular body to invert itself. Specifically, the malleable generally tubular body includes an exterior portion and an interior portion defining a sealed compartment configured to maintain a biocompatible fluid therein. When the sleeve is rolled or rolling, the exterior and interior portions repeatedly invert as the malleable generally tubular body is moved longitudinally.

The methods described herein may further include either or both of steps 230 and 240. In some embodiments, the method further include performing at least one of a treating, testing, or biopsying step either with or through the endoluminal shaft 230. In some embodiments, the method further includes withdrawing the one or more rolling sleeves, endoluminal shaft, and any optional anchor members from the body lumen 240.

In some embodiments, the step of positioning the endoluminal shaft into the sleeve may include the use of an endoluminal robotic shaft.

In some embodiments, the step of advancing the distal end portion of the endoluminal shaft and the protective rolling sleeve longitudinally into the body lumen may include the use of an endoluminal robotic shaft.

In some embodiments, the step of withdrawing the distal end portion of the endoluminal shaft and the protective rolling sleeve longitudinally out of the body lumen may include the use of an endoluminal robotic shaft.

In some embodiments, the step of advancing the distal end portion of the endoluminal shaft and the protective rolling sheet longitudinally into the body lumen may cause the exterior portion of the tubular body to move to an interior part of the tubular body and the interior portion of the tubular body to become an exterior part of the tubular body.

In some embodiments, the methods may further include positioning one or more anchor members on the endoluminal shaft before positioning the distal end of the endoluminal shaft into the channel defined within the malleable generally tubular body of the protective rolling sleeve.

In some embodiments, the methods may further include positioning one or more anchor members on the endoluminal shaft after positioning the distal end of the endoluminal shaft into the channel defined within the malleable generally tubular body of the protective rolling sleeve.

In some embodiments, the endoluminal shaft may further include one or more inflatable anchor members positioned thereon, the inflatable anchor members configured to at least maintain the position of the rolling sleeve relative to the endoluminal shaft. The one or more inflatable anchor members may also be configured to advance the rolling sleeve longitudinally relative to the endoluminal shaft.

The sleeves described herein may be formed using any suitable method, including but not limited to, extrusion, pressing, molding, casting, and the like. In some embodiments, the sleeves may be formed by an extrusion or molding process.

In some embodiments, the sleeve may be formed by a blow-molding process which creates a tube outer shape which can receive a fluid therein prior to sealing of the tube. Then pushing a rod through the center of the tube longitudinally and cutting/sealing the end to create a channel or lumen therethrough.

In some embodiments, the sleeve may be formed by an extrusion process which creates either a tube outer shaped body or a plastic sheet that can be rolled into a tube outer shaped body which can receive a fluid therein prior to heat-sealing of the tube. Then pushing a rod through the center of the tube longitudinally and cutting/sealing the end to create a channel or lumen therethrough.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting but merely as exemplifications of particular embodiments. Those skilled in the art will envision other modifications within the scope and spirit of the claims appended hereto.

What is claimed is:

1. A rolling sleeve configured for positioning on an endoluminal shaft comprising:
a malleable generally tubular body including an exterior portion and an interior portion defining a sealed compartment, the tubular body defining a channel therethrough along the interior portion, the channel configured to receive at least a distal end portion of an endoluminal shaft therein; and
a biocompatible fluid maintained in the sealed compartment, wherein the exterior and interior portions are configured to repeatedly invert as the malleable generally tubular body is moved longitudinally.

2. The rolling sleeve of claim 1, wherein the generally tubular body is formed of an elastic biocompatible material.

3. The rolling sleeve of claim 1, wherein the biocompatible fluid is a liquid selected from the group consisting of water, saline, dextrose, lactated ringers, and combinations thereof.

4. The rolling sleeve of claim 1, wherein the biocompatible fluid is a gel.

5. The rolling sleeve of claim 1, wherein the channel is a malleable channel.

6. The rolling sleeve of claim 1, wherein the generally tubular body defines a generally circular transverse cross-section.

7. The rolling sleeve of claim 1, wherein the generally tubular body defines a generally non-circular transverse cross-section.

8. The rolling sleeve of claim 7, wherein the generally tubular body further comprises one or more exterior pleats extending along an entire length of an exterior of the tubular body, the one or more exterior pleats configured to create and maintain one or more external air gaps between the exterior of the tubular body and a body lumen positioned along the exterior to allow air to pass therethrough while positioned within the body lumen.

9. The rolling sleeve of claim 8, wherein the generally tubular body further comprises one or more interior pleats extending along an entire length of the channel of the tubular body, the one or more interior pleats configured to create and maintain one or more internal air gaps between an exterior of the channel and an exterior of the shaft to allow air to pass therethrough.

10. The rolling sleeve of claim 9, wherein the one or more pleats define a non-circular transverse cross-section selected from the group consisting of a heart-shaped cross-section, an X-shaped cross-section, a Y-shaped cross-section, or a flower petal cross-section.

11. The rolling sleeve of claim 1, further comprising at one of a plurality of wireless electrodes, a plurality of wireless pressure sensors, or both.

12. A method of inserting an endoluminal shaft into a body lumen, the method comprising:
positioning a distal end portion of the endoluminal shaft into a channel defined within a malleable generally tubular body of a rolling sleeve, the malleable generally tubular body including an exterior portion and an interior portion defining a sealed compartment, a biocompatible fluid maintained in the sealed compartment, wherein the exterior and interior portions are configured to repeatedly invert as the malleable generally tubular body is moved longitudinally; and
advancing the distal end portion of the endoluminal shaft and the rolling sheet longitudinally into a body lumen.

13. The method of claim 12, wherein advancing the distal end portion of the endoluminal shaft and the rolling sleeve longitudinally into the body lumen causes the exterior portion of the tubular body to move to an interior part of the tubular body and the interior portion of the tubular body to become an exterior part of the tubular body.

14. The method of claim 12, further comprising positioning one or more anchor members on the endoluminal shaft before positioning the distal end of the endoluminal shaft into the channel defined within the malleable generally tubular body of the rolling sleeve.

15. The method of claim 12, further comprising positioning one or more anchor members on the endoluminal shaft after positioning the distal end of the endoluminal shaft into the channel defined within the malleable generally tubular body of the rolling sleeve.

16. The method of claim 12, wherein the endoluminal shaft further comprises one or more inflatable anchor members positioned thereon, the inflatable anchors configured to at least maintain the position of the rolling sleeve relative to the endoluminal shaft and optionally move the rolling sleeve longitudinally relative to the endoluminal shaft.

17. The method of claim 12, wherein the endoluminal shaft is an endoluminal robotic shaft.

18. The method of claim 12, wherein the endoluminal shaft is an Endoflip™ Catheter.

19. The method of claim 12, wherein the endoluminal shaft is an Esoflip™ Catheter.

20. A kit comprising:
one or more rolling sleeves, each including a malleable generally tubular body including an exterior portion and an interior portion defining a sealed compartment, the tubular body defining a channel therethrough along the interior portion, the channel configured to receive an endoscope therein; and a biocompatible fluid maintained in the sealed compartment, wherein the exterior and interior portions are configured to repeatedly invert as the malleable generally tubular body is moved longitudinally: and
one or more anchor members or one or more endoluminal shafts including one or more inflatable anchor members positioned thereon.
